(19) United States
(12) Patent Application Publication
(10) Pub. No.: US 2026/0152617 A1
(43) Pub. Date: Jun. 4, 2026

(54) METHODS FOR DEPOLYMERIZATION OF SUBSTITUTED VINYL POLYMERS

(71) Applicant: BOARD OF TRUSTEES OF MICHIGAN STATE UNIVERSITY, East Lansing, MI (US)

(72) Inventors: Muhammad Rabnawaz, Okemos, MI (US); Ajmir Khan, East Lansing, MI (US); Vikash Kumar, East Lansing, MI (US)

(21) Appl. No.: 18/695,876

(22) PCT Filed: Oct. 4, 2022
(86) PCT No.: PCT/US2022/045657

§ 371 Date: Mar. 27, 2024

Related U.S. Application Data
(60) Provisional application No. 63/252,286, filed on Oct. 5, 2021.

(51) Int. Cl. C08J 11/16; C07C 4/22; C07C 7/04; C07C 7/20

(52) U.S. Cl. CPC C08J 11/16; C07C 4/22; C07C 7/04; C07C 7/20

(57) ABSTRACT

The disclosure relates to a method for depolymerizing a substituted vinyl polymer, such as polystyrene. The method can form part of a chemical recycling process to depolymerize a vinyl polymer feed into desired monomers or oligomers, which can then be used to re-polymerize and re-form new polymer materials. The method includes heating a mixture of a vinyl polymer having a pendant aromatic or ester group and a metal or carbon-carbon network catalyst. The heating is performed at a temperature above a melting temperature of the vinyl polymer, but low enough such that the catalyst is in solid form in the mixture. The product resulting from depolymerization and heating can include one or both of a corresponding monomer product and a corresponding oligomer product from the original vinyl polymer.

### CROSS REFERENCE TO RELATED APPLICATION

Priority is claimed to U.S. Provisional Application No. 63/252,286 filed on Oct. 5, 2021, which is incorporated herein by reference in its entirety.

### STATEMENT OF GOVERNMENT INTEREST

None.

### BACKGROUND OF THE DISCLOSURE

### Field of the Disclosure

The disclosure relates to a method for depolymerizing a substituted vinyl polymer, such as polystyrene, for example as part of a chemical recycling process. The method includes heating a mixture of a vinyl polymer having a pendant aromatic or ester group and a solid metal or carbon-carbon network catalyst, thereby depolymerizing the vinyl polymer to form corresponding monomers and/or oligomers from the vinyl polymer.

### Brief Description of Related Technology

Over 80 billion pounds (or 36.4 billion kg) of plastics are produced in the US alone each year. Approximately 85% of the plastics generated worldwide end up in landfills or incinerators, and plastics waste poses a threat to ecosystems due to microplastics and the release of hazardous substances.

Plastic waste materials are currently recycled via four different approaches: primary recycling (e.g., mechanical recycling for the same purpose), secondary recycling (e.g., often for down-cycled applications), tertiary or chemical recycling, and quaternary recycling for energy recovery (e.g., incineration). Polymers recycled by primary and secondary recycling cannot be recycled for multiple cycles due to oxidative degradations.

Polystyrene (PS) is annually produced at a scale of more than 22 million tons worldwide. PS is typically used for the production of appliances, automobile parts, electronics, gardening pots, toys, equipment, food-service items, and so forth, often in combination with various colorants, other plastics, or additives. PS foams are widely used for packaging applications. Unfortunately, only negligible amounts of PS are treated via chemical recycling because the development of effective methods for chemical recycling of PS back to its monomers presents a daunting technological challenge. Key problems include: 1) the thermal degradation of PS yields 10 to 20 various small molecules and oligomeric substances; 2) lower yields of styrene from PS, and 3) longer reaction time. Previous studies are dedicated to fluidized bed reactors and the use of solvents to overcome some of these challenges.

### SUMMARY

In one aspect, the disclosure relates to a method for depolymerizing a substituted vinyl polymer, the method comprising: heating a mixture comprising (i) a vinyl polymer (or copolymer) comprising one or more vinyl monomer units comprising at least one of a pendant aromatic group and a pendant ester group (e.g., a vinyl (co)polymerization product thereof, such as an olefinic backbone with pendant aromatic and/or ester groups) and (ii) a catalyst comprising at least one of a metal catalyst and a carbon-carbon network catalyst to a temperature above a melting temperature of the vinyl polymer and (optionally) below a decomposition temperature of the vinyl polymer, thereby at least partially depolymerizing the vinyl polymer and forming at least one of a corresponding monomer product and a corresponding oligomer product (e.g., oligomers with 2, 3, 4, 5, 6, 7, 8, 9, or 10 monomer units). The catalyst is in solid form in the mixture at the temperature to which the mixture is heated (e.g., also in solid form as initially added to the mixture; no melting or formation of a liquid catalyst medium).

Various refinements of the disclosed methods are possible.

In a refinement, the vinyl polymer comprises an aromatic polymer comprising one or more vinyl monomer units comprising a pendant aromatic group.

In a further refinement, the aromatic polymer comprises polystyrene. For example, depolymerizing the aromatic polymer can comprise forming styrene as the corresponding monomer product. Alternatively or additionally, depolymerizing the aromatic polymer comprises forming one or more styrene oligomers as the corresponding oligomer product.

In a further refinement, the aromatic polymer comprises a styrene copolymer (e.g., styrene-butadiene rubber (SBR), acrylonitrile butadiene styrene (ABS), styrene-isoprene-styrene (SIS), styrene-ethylene/butylene-styrene (S-EB-S), styrene-divinylbenzene (S-DVB), styrene-acrylonitrile, acrylonitrile styrene acrylate (ASA), styrene (meth)acrylate, styrene (meth)acrylic acid).

In a further refinement, the aromatic polymer comprises polymethylstyrene; and depolymerizing the aromatic polymer comprises forming at least one of «-methylstyrene as the corresponding monomer product and one or more α-methylstyrene oligomers as the corresponding oligomer product.

In a further refinement, the aromatic polymer comprises monomer (or repeat) units resulting from (free radical) polymerization of monomers according to the general formula CH₂═CR¹R²; and R¹ and R² independently can be H, substituted or unsubstituted C₁-C₆ (e.g., having 1, 2, 3, 4, 5, or 6 carbon atoms) linear or branched alkyl groups, substituted or unsubstituted C₂-C₈ (e.g., having 2, 3, 4, 5, 6, 7, or 8 carbon atoms) linear or branched (alkyl) ester groups, substituted or unsubstituted phenyl groups, substituted or unsubstituted fused polyaromatic groups (e.g., naphthyl). In such cases, at least one of R¹ and R² includes an aromatic group (e.g., R¹═H and R²=phenyl corresponds to styrene, R¹═CH₃ and R²=phenyl corresponds to α-methylstyrene, R¹═H and R²=naphthyl corresponds to vinyl naphthalene, etc.).

In a refinement, the vinyl polymer comprises one or more vinyl monomer units comprising a pendant ester group. In a further refinement, the vinyl polymer comprises monomer (or repeat) units resulting from (free radical) polymerization of monomers according to the general formula CH₂═CR¹R²; and R¹ and R² independently can be H, substituted or unsubstituted C₁-C₆ (e.g., having 1, 2, 3, 4, 5, or 6 carbon atoms) linear or branched alkyl groups, substituted or unsubstituted C₂-C₈ (e.g., having 2, 3, 4, 5, 6, 7, or 8 carbon atoms) linear or branched (alkyl) ester groups, substituted or unsubstituted phenyl groups, substituted or unsubstituted fused polyaromatic groups (e.g., naphthyl); with the proviso that at least one of R¹ and R² includes an ester group (e.g., R¹═H and R²═—C(═O)O—CH₃ corresponds to methyl acrylate, R¹═CH₃ and R²═—C(═O)O—CH₃ corresponds to methyl methacrylate, etc.).

In a refinement, the vinyl polymer is selected from the group consisting of homopolymers, copolymers (e.g., 2, 3, or more different monomer units in block and/or random arrangement), and combinations (e.g., blends) thereof.

In a refinement, the method comprises depolymerizing the vinyl polymer with a conversion of at least 70 wt. % (e.g., at least 70, 75, 80, 82, 85, 87, 90, or 92 wt. % and/or up to 85, 88, 90, 92, 95, 98, or 99 wt. % of original vinyl polymer is converted to and/or recovered as the corresponding monomer and/or oligomer products combined, such as in a liquid product phase; remainder of unconverted vinyl polymer can be present after reaction as a solid phase, such as unreacted vinyl polymer and/or carbon-based burning/combustion/etc. residue).

In a refinement, depolymerizing the vinyl polymer comprises forming a liquid product phase containing the corresponding monomer product in an amount of at least 75 wt. % relative to the liquid product phase (e.g., at least 75, 77, 80, 82, 85, 87, 90, or 92 wt. % and/or up to 85, 88, 90, 92, 95, 98, or 99 wt. % of the liquid product phase is the corresponding monomer or monomers from the original vinyl polymers; the foregoing ranges can similarly represent monomer yield (relative amount of original vinyl polymer converted to and/or recovered as monomer), such as where there is substantially complete conversion of the vinyl polymer reactant).

In a further refinement, the corresponding monomer product is formed with a selectivity of at least 6 relative to other ethylenically unsaturated products (e.g., selectivity of at least 6, 8, 10, 12, 15, 20, 30, or 40 and/or up to 15, 20, 30, 50, 100, or 200 on a weight/weight basis; ethylenically unsaturated products can include ethylenically unsaturated oligomers, ethylenically unsaturated monomers different from the monomer(s) in the original vinyl polymer, etc.).

In a further refinement, depolymerizing the vinyl polymer comprises forming the corresponding oligomer product in an amount of 25 wt. % or less relative to the liquid product phase (e.g., at least 0.1, 1, 2, 3, or 5 wt. % and/or up to 1, 2, 5, 7, 10, 12, 15, 20, or 25 wt. % of the liquid product phase is the one or more corresponding oligomer products with variable lengths, such as dimers, trimers, or higher oligomers, in particular oligomers with 2-4 repeat units; liquid product phase can further include up to 25 wt. % (with similar subranges) of unsaturated analogs of the corresponding monomers and/or oligomers).

In a refinement, depolymerizing the vinyl polymer comprises forming a liquid depolymerization product (e.g., an essentially completely depolymerized product such as described above with high monomer yield; alternatively a partially depolymerized product to be liquid at room temperature but still containing substantial amounts of oligomeric chains).

In a further refinement, depolymerizing the vinyl polymer comprises forming the corresponding monomer product in an amount of 50 wt. % or less relative to the liquid depolymerization product (e.g., at least 0.1, 1, 2, 3, 5, 10, 15, 20, 25, 30, or 35 wt. % and/or up to 1, 2, 5, 10, 20, 30, 40, or 50 wt. % of the liquid depolymerization product is the corresponding monomer product; liquid depolymerization product can further include up to 25 wt. % (with similar subranges) of unsaturated analogs of the corresponding monomers and/or oligomers); and depolymerizing the vinyl polymer comprises forming the corresponding oligomer product in an amount of at least 50 wt. % relative to the liquid depolymerization product (e.g., at least 50, 60, 70, 75, 80, 85, 90, or 95 wt. % and/or up to 65, 75, 85, 90, 95, 98, 99, or 100 wt. % of the liquid depolymerization product is the one or more corresponding oligomer products with variable lengths, such as dimers, trimers, or higher oligomers, in particular oligomers with 2-4 repeat units).

In a refinement, depolymerizing the vinyl polymer comprises forming the corresponding monomer product; and the method further comprises polymerizing the corresponding monomer product (e.g., at a suitable (heated) temperature after addition of an azo, organic peroxide, aminoxyl, or other free radical initiator) to form a recycled vinyl polymer.

In a refinement, the catalyst is present in the mixture in an amount of 0.5 wt. % to 10 wt. %, 20 wt. %, 35 wt. %, 50 wt. % or 100 wt. % (e.g., at least 0.5, 1, 2, 3, 4, 5, 7, 10, 20, 30, 40, or 50 wt. % and/or up to 4, 5, 6, 7, 8, 9, 10, 20, 35, 50, 75, or 100 wt. %).

In a refinement, the catalyst comprises a metal catalyst selected from the group consisting of metal salts, metal oxides, elemental metals, metal alloys, and combinations thereof (e.g., including one or more alkali metals such as Li, Na, K; alkaline earth metals such as Be, Ca, Mg, Ba; transition metals such as Ti, V, Cr, Mn, Fe, Co, Ni, Cu, Zn; salts thereof such as halide salts, acetate or ester salts; oxides thereof, alloys thereof such as steel or stainless steel; solid form such as powder, mesh, wire).

In a further refinement, the metal catalyst comprises an alkali metal halide salt catalyst selected from sodium chloride and potassium chloride (preferably sodium chloride).

In a further refinement, the metal catalyst comprises an alkali metal halide salt catalyst comprising: at least one of a lithium cation, a sodium cation, and a potassium cation; and at least one of a fluoride anion, chloride anion, bromide anion, and iodide anion.

In a refinement, the catalyst comprises a carbon-carbon network (e.g., 2D network, 3D network) catalyst selected from the group consisting of graphite, graphene nanoplatelets, carbon nanotubes (e.g., single walled, multi-walled), and combinations thereof.

In a refinement, the method comprises depolymerizing the vinyl polymer by heating the mixture to a temperature in a range of 250° C. to 400° C., 500° C., or 600° C. (e.g., at least 250, 280, 300, 320, 340, 360, 380, 400, 450, 500, or 550° C. and/or up to 320, 340, 360, 380, 400, 420, 450, 500, 550, or 600° C.; upper temperature bound is also below a temperature that would melt the catalyst, for example single catalyst, mixture of catalysts, eutectic mixture of catalysts, etc.).

In a refinement, the method comprises depolymerizing the vinyl polymer (e.g., polystyrene) by heating the mixture to a temperature in a range of 300° C. to 420° C. (e.g., 300° C. or 340° C. to 340° C., 380° C., or 420° C.).

In a refinement, the method comprises depolymerizing the vinyl polymer by heating the mixture to: (i) a temperature at least 10° C. (e.g., at least 10, 20, 30, or 50° C.) above a melting temperature of the vinyl polymer; and/or (ii) a temperature at least 50° C. (e.g., at least 10, 20, 30, 40, 50, 70, or 100° C.) below the decomposition temperature of the vinyl polymer (e.g., where the decomposition temperature of the vinyl polymer can represent the lower of the temperature at which the vinyl polymer would depolymerize in the absence of a catalyst or other additive (pyrolysis temperature) and the temperature at which the vinyl polymer would combust, burn, char, etc. to form a carbon-based product other than monomer).

In a refinement, the method comprises depolymerizing the vinyl polymer by heating the mixture to a temperature up to 200° C. (e.g., up to 10, 20, 30, 40, 50, 70, 100, 150, or 200° C.) above the decomposition temperature of the vinyl polymer (e.g., where such temperatures can further accelerate depolymerization to form monomer and/or oligomer, but without substantial combustion, burning, charring, etc. that would destroy the monomer/oligomer products).

In a refinement, the vinyl polymer is initially in a form selected from foamed materials, un-foamed materials, and combinations thereof (e.g., prior to heating and/or prior to addition to the mixture).

In a refinement, the vinyl polymer (e.g., polystyrene) is present in the mixture in an amount of 80 wt. % or 90 wt. % to 99 wt. % (e.g., at least 80, 82, 84, 86, 88, 90, 92, 94, or 96 wt. % and/or up to 90, 92, 94, 95, 96, 97, 98, or 99 wt. %).

In a refinement, a combined amount of the vinyl polymer and the catalyst in the mixture is in a range of 80 wt. % or 90 wt. % to 100 wt. % (e.g., at least 80, 82, 84, 86, 88, 90, 92, 94, 96, 98, or 99 wt. % and/or up to 90, 92, 94, 95, 96, 97, 98, 99, 99.5, or 100 wt. %, for example including all polymer reactants and all catalysts when there is more than one species of polymer and/or catalyst components in the initial reaction mixture).

In a refinement, the mixture further comprises one or more additives or impurities in a (combined) amount up to 20 wt. % (e.g., one or more of salts, metal residues, organic impurities or additives, inorganic impurities or additives, moisture, plastic additives, etc. that might be present in a recycled feed as impurities and/or added to the feed mixture as a processing aid; such as at least 0.01, 0.1, 1, 2, or 5 wt. % and/or up to 0.5, 1, 2, 3, 5, 8, 12, 15, or 20 wt. %).

In a refinement, the mixture further comprises one or more low-melting thermoplastic polymers in a (combined) amount up to 20 wt. % (e.g., polyolefins such as polyethylene (PE, LDPE, HDPE, etc.), polypropylene, etc. that typically have a melting point below about 200° C. or otherwise well below that of the vinyl polymer such that the low-melting thermoplastic polymer melts during heating to provide an initial liquid polymer phase to promote vinyl polymer-catalyst contact and enhance vinyl polymer conversion/yield; the low-melting thermoplastic polymer can be an intentional additive and/or impurity present from a given waste stream to be recycled; such as at least 0.01, 0.1, 1, 2, or 5 wt. % and/or up to 0.5, 1, 2, 3, 5, 8, 12, 15, 20, 30, or 50 wt. %).

In a refinement, the mixture further comprises one or more free radical inhibitors or suppressors in a (combined) amount up to 5 wt. % (e.g., at least 0.01, 0.1, 1, or 2 wt. % and/or up to 0.5, 1, 2, 3, 4, or 5 wt. %).

While the disclosed articles, apparatus, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### DETAILED DESCRIPTION

The disclosure relates to a method for depolymerizing a substituted vinyl polymer, such as polystyrene (PS). The method can form part of a chemical recycling process to depolymerize a PS or other polymer feed into desired monomers or oligomers, which can then be used to re-polymerize and re-form new (virgin) polymer materials. The disclosed method provides a high conversion and yield not obtainable by other chemical recycling methods, for example above 80% for styrene formed from a PS feedstock. The method utilizes relatively inexpensive depolymerization catalysts in small amounts, for example alkali metal halides such as sodium chloride. The depolymerization catalysts are inexpensive and non-toxic, and the catalysts can be re-used in multiple successive depolymerization processes without being depleted, etc. While the examples below illustrate the method for the particular case of PS, the disclosed method is applicable to other substituted vinyl polymer as well, such as poly(methyl methacrylate) (PMMA), polymethylstyrene, etc.

More generally, the disclosure relates to a method for depolymerizing a substituted vinyl polymer, which includes heating a mixture including a vinyl polymer or copolymer having a pendant aromatic or pendant ester group and a metal catalyst or a carbon-carbon network catalyst. The catalyst is in solid form in the mixture at the temperature to which the mixture is heated, for example with the catalyst also being in solid form as initially added to the mixture. More generally, there is no melting of the catalyst or formation of a liquid catalyst medium during heating in particular and/or in the process more generally. The heating is performed at or to a temperature above a melting temperature of the vinyl polymer and (optionally) below a decomposition temperature of the vinyl polymer, thereby at least partially depolymerizing the vinyl polymer. The product resulting from depolymerization and heating can include one or both of a corresponding monomer product and a corresponding oligomer product, such as oligomers with 2, 3, 4, 5, 6, 7, 8, 9, and/or 10 monomer units. For example, depolymerization of polystyrene as the vinyl polymer according to the disclosed method can provide styrene as the corresponding monomer product, and styrene dimers, trimers, etc. as the corresponding oligomer product.

### Vinyl Polymers

The vinyl polymer or copolymer includes one or more vinyl monomer units with a pendant aromatic group and a pendant ester group, for example including a vinyl (co)polymerization product thereof. For example, the vinyl polymer can have an olefinic backbone with pendant aromatic and/or ester groups. The term “vinyl polymer” is used herein interchangeably with a vinyl copolymer, and it can include, for example, vinyl homopolymers in which all of the monomers have one or both of a pendant aromatic group and a pendant ester group, vinyl copolymers in which all of the different monomers have a pendant aromatic group and/or a pendant ester group, vinyl copolymers in which some monomers have a pendant aromatic group and/or a pendant ester group and some monomers have no pendant aromatic or ester groups. In embodiments, the vinyl polymer can include one or more of vinyl homopolymers and vinyl copolymers (e.g., 2, 3, or more different monomer units in block and/or random arrangement), for example in mixtures or blends when there is more than one vinyl polymer.

The vinyl polymer can generally include monomer (or repeat) units resulting from polymerization (e.g., free radical polymerization) of monomers according to the general formula CH₂═CR¹R², which can similarly represent a depolymerization monomer product of the disclosed method. Accordingly, a corresponding monomer in the vinyl polymer can be represented by —[CH₂—C(R¹R²)]— as a repeat unit, which can similarly represent a monomer unit in a depolymerization oligomer product of the disclosed method. R¹ and R² independently can be H, substituted or unsubstituted C₁-C₆ (e.g., having 1, 2, 3, 4, 5, or 6 carbon atoms) linear or branched alkyl groups, substituted or unsubstituted C₂-C₈ (e.g., having 2, 3, 4, 5, 6, 7, or 8 carbon atoms) linear or branched (alkyl) ester groups, substituted or unsubstituted phenyl groups, or substituted or unsubstituted fused polyaromatic groups (e.g., naphthyl). For the vinyl monomer units having a pendant aromatic or ester group, at least one of R¹ and R² includes an aromatic group (e.g., when the vinyl polymer has a pendant aromatic group) and/or an ester group (e.g., when the vinyl polymer has a pendant ester group). As examples of vinyl polymers and corresponding monomers with pendant aromatic groups, R¹═H and R²=phenyl corresponds to styrene, R¹═CH₃ and R²=phenyl corresponds to α-methylstyrene, R¹═H and R²=naphthyl corresponds to vinyl naphthalene, etc. As examples of vinyl polymers and corresponding monomers with pendant ester groups, R¹═H and R²═—C(═O)O—CH₃ corresponds to methyl acrylate, R¹═CH₃ and R²═—C(═O)O—CH₃ corresponds to methyl methacrylate, etc.

In embodiments, the vinyl polymer can include comonomer (or repeat) units without a pendant aromatic or ester group. Such comonomers can similarly be represented by the above formula CH₂═CR¹R², but without a requirement that at least one of R¹ and R² includes an ester or aromatic group. For example, R¹═H and R²═H corresponds to ethene as a comonomer, R¹═H and R²═CH₃ corresponds to propene as a comonomer, R¹═H and R²═CH₂CH₃ corresponds to 1-butene as a comonomer, etc. Other comonomers can include one or more of butadiene, acrylonitrile, isoprene, divinylbenzene (e.g., a di-vinyl-functional aromatic monomer), etc.

In embodiments, the vinyl polymer is an aromatic polymer including one or more vinyl monomer units having a pendant aromatic group. An example includes styrene, such as in polystyrene for the corresponding polymer. In particular embodiments, the disclosed depolymerization method can include forming styrene as the corresponding monomer product and/or one or more styrene oligomers as the corresponding oligomer product. Another example includes α-methylstyrene, such as in polymethylstyrene for the corresponding polymer. In particular embodiments, the disclosed depolymerization method can include forming α-methylstyrene as the corresponding monomer product and/or one or more α-methylstyrene oligomers as the corresponding oligomer product.

In embodiments, the aromatic polymer can include a styrene copolymer. Examples include styrene-butadiene rubber (SBR), acrylonitrile butadiene styrene (ABS), styrene-isoprene-styrene (SIS), styrene-ethylene/butylene-styrene (S-EB-S), styrene-divinylbenzene (S-DVB), and styrene-acrylonitrile. Further examples include styrene copolymers that also have pendant ester groups as well, such as acrylonitrile styrene acrylate (ASA), styrene (meth)acrylate, and styrene (meth)acrylic acid.

In embodiments, the vinyl polymer includes one or more vinyl monomer units having a pendant ester group. Examples of such monomer units include methyl acrylate (e.g., with corresponding polymers thereof such as poly(methyl acrylate)) and methyl methacrylate (e.g., with corresponding polymers thereof such as poly(methyl methylacrylate)).

The vinyl polymer to be depolymerized can initially be in a variety of forms, for example depending on the feedstock being depolymerized (e.g., original plastic-containing product being recycled). For example the vinyl polymer can initially be in the form of foamed materials, un-foamed materials, and mixtures thereof (e.g., a mixture of particles or fragments of some foamed materials and some un-foamed materials).

The amount of the vinyl polymer in the mixture or feedstock to be depolymerized is not particularly limited, and it can vary widely depending on the desired catalyst loading, the potential presence of impurities, the potential inclusion of processing aids or additives, etc. In embodiments, the vinyl polymer can be present in the mixture in an amount of 50-99 wt. %, 80-99 wt. %, or 90-99 wt. %, for example based on the total weight/amount of mixture (e.g., combined amount of vinyl polymer, catalyst, impurities, additives, etc.). For example, the vinyl polymer can be present in the mixture in an amount of at least 50, 60, 70, 80, 82, 84, 86, 88, 90, 92, 94, or 96 wt. % and/or up to 65, 80, 90, 92, 94, 95, 96, 97, 98, or 99 wt. %. Alternatively or additionally, a combined amount of the vinyl polymer(s) and the catalyst(s) in the mixture can be present in the mixture in an amount of 80-100 wt. % or 90-100 wt. %, similarly based on the total weight/amount of mixture (e.g., combined amount of vinyl polymer, catalyst, impurities, additives, etc.). For example, the vinyl polymer(s) and the catalyst(s) can be present in the mixture in a combined amount of at least 80, 82, 84, 86, 88, 90, 92, 94, 96, 98, or 99 wt. % and/or up to 90, 92, 94, 95, 96, 97, 98, 99, 99.5, or 100 wt. %, for example including all vinyl polymer reactants (e.g., with pendant aromatic and/or ester groups) and all catalysts combined when there is more than one species of vinyl polymer and/or catalyst components in the initial reaction mixture.

### Catalysts

The primary catalyst in the mixture to be heated includes at least one of a metal catalyst and a carbon-carbon network catalyst to promote depolymerization of the vinyl polymer into its constituent corresponding monomers and/or oligomers.

Examples of metal catalysts include metal salts, metal oxides, elemental metals, metal alloys, and combinations or mixtures thereof. Such metals in the various forms can include one or more alkali metals such as Li, Na, K; alkaline earth metals such as Be, Ca, Mg, Ba; transition metals such as Ti, V, Cr, Mn, Fe, Co, Ni, Cu, Zn; salts thereof such as halide salts, acetate or ester salts; oxides thereof; and alloys thereof such as steel or stainless steel. The metal catalyst is generally in a solid form such as powder, mesh, wire, etc.

In embodiments, the metal catalyst can include an alkali metal halide salt, for example including at least one of a lithium cation, a sodium cation, and a potassium cation; and at least one of a fluoride anion, chloride anion, bromide anion, and iodide anion. In particular embodiments, the metal catalyst can include sodium chloride and/or potassium chloride.

Examples of carbon-carbon network catalysts include those with 2-dimensional or 3-dimensional carbon-carbon networks or lattices, for example graphite, graphene nanoplatelets, single walled carbon nanotubes, multi-walled carbon nanotubes, and combinations or mixtures thereof.

The catalyst can be present in the mixture during heating and depolymerization in relatively wide ranges, although it can be particularly effective in terms of relatively high depolymerization conversion or yield even at relatively low concentrations. In embodiments, the catalyst can be present in the mixture in an amount of 0.5 wt. % to 10 wt. %, 20 wt. %, 35 wt. %, 50 wt. % or 100 wt. % relative to the (total) vinyl polymer present originally present in the mixture. For example, the catalyst (e.g., all metal catalysts and all carbon-carbon network catalysts combines) can be present in amount of at least 0.5, 1, 2, 3, 4, 5, 7, 10, 20, 30, 40, or 50 wt. % and/or up to 4, 5, 6, 7, 8, 9, 10, 20, 35, 50, 75, or 100 wt. % relative to the (total) vinyl polymer in the mixture. In embodiments, the catalyst can be present in the mixture in an amount of 0.5 wt. % to 10 wt. %, 20 wt. %, 35 wt. %, or 50 wt. %, for example based on the total weight/amount of mixture (e.g., combined amount of vinyl polymer, catalyst, impurities, additives, etc.). For example, the catalyst can be present in the mixture in an amount of at least 0.5, 1, 2, 3, 4, 5, 7, 10, or 20 wt. % and/or up to 3, 4, 5, 6, 7, 8, 9, 10, 20, 35, or 50 wt. %.

### Depolymerization and Products

Heating of the mixture to effect depolymerization can be performed in any suitable reaction vessel (e.g., batch, continuous, etc.) up to or at a temperature suitably selected for the particular vinyl polymer(s) being depolymerized and the particular catalyst(s) being incorporated into the mixture. For example, the depolymerization reaction can be performed in a batch or continuous distillation apparatus such that such that the high mixture temperature required to melt the vinyl polymer and effect depolymerization will also vaporize formed monomer and other low-boiling oligomers or byproducts, which in turn can be separated from the liquid reaction mixture (i.e., as a separate phase) and recovered as a liquid product after condensation or other cooling. As described above, the heating or depolymerization temperature is above the melting temperature of the vinyl polymer in the mixture, for example being above the highest melting temperature in a mixture with multiple different vinyl polymers, thereby providing a bulk liquid/polymer melt reaction medium for depolymerization. Additionally, the heating or depolymerization temperature is low enough such that the catalyst (e.g., metal catalyst and/or carbon-carbon network catalyst) remains in solid form (e.g., powder, particulate, etc.) in the mixture, for example being below the lowest melting and/or decomposition temperature in a mixture with one or more different catalysts (e.g., single catalyst, mixture of catalysts, eutectic mixture of catalysts), such that the catalyst can be a discrete or separate solid phase in the bulk liquid/polymer melt reaction medium for depolymerization. During depolymerization, suitable reaction pressures can be ambient pressure or approximately atmospheric pressure, for example 0.2-5 bar, 0.5-1.5 bar, 0.8-1.2 bar, or 0.9-1.1 bar, which can represent a pressure in one or more of the reaction vessel where depolymerization occurs, a product vessel where monomer and/or oligomer product is collected (e.g., a receiving flask or other vessel to collect depolymerization product condensate as a liquid), etc. Suitable reaction times can be selected as appropriate based on desired conversion the reactant, selectivity of the desired product (e.g., monomer vs. oligomer or monomer/oligomer vs. other byproducts), etc. for example 0.05-5 hr, 0.2-2 hr, 0.5-1.5 hr, or 0.8-1.2 hr.

In embodiments, heating of the mixture to effect depolymerization can be performed up to or at a temperature in a range of 250° C.-400° C., 250° C.-500° C., or 250° C.-600° C., for example representing an essentially constant depolymerization temperature in the range or a variable-temperature profile in which the maximum temperature is within the range. More generally, the depolymerization temperature can be at least 250, 280, 300, 320, 340, 360, 380, 400, 450, 500, or 550° C. and/or up to 320, 340, 360, 380, 400, 420, 450, 500, 550, or 600° C., depending on the particular vinyl polymer(s) in the mixture. In the particular case of polystyrene as at least one of the vinyl polymers, the particularly suitable depolymerization temperatures can be in a range of 300° C. to 420° C. (e.g., at least 300° C. or 340° C. and/or up to 340° C., 380° C., or 420° C.).

In embodiments, heating of the mixture to effect depolymerization can be performed up to or at a temperature that is at least 10° C. above a melting temperature of the vinyl polymer, and/or a temperature that is at least 50° C. below the decomposition temperature of the vinyl polymer. For example, the depolymerization temperature can be at least 10, 20, 30, or 50° C. above the melting temperature of the vinyl polymer, such as above the highest melting temperature in a mixture with multiple different vinyl polymers.

Alternatively or additionally, the depolymerization temperature can be at least 10, 20, 30, 40, 50, 70, or 100° C. below the decomposition temperature of the vinyl polymer, such as below the lowest decomposition temperature in a mixture with multiple different vinyl polymers. The decomposition temperature of the vinyl polymer can represent the lower of the temperature at which the vinyl polymer would depolymerize in the absence of a catalyst or other additive (pyrolysis temperature) and the temperature at which the vinyl polymer would combust, burn, char, etc. to form a carbon-based product other than monomer.

While depolymerization is typically performed below the decomposition temperature of the vinyl polymer, the depolymerization temperature in some embodiments can be selected to be above the decomposition temperature of one or more vinyl polymers in the mixture. Such elevated temperatures can further accelerate depolymerization to form monomer and/or oligomer, but are selected to be not so excessive that they would otherwise results in substantial combustion, burning, charring, etc. that would destroy the monomer/oligomer products. For example, the depolymerization temperature can be at a temperature up to 200° C. above the decomposition temperature of the vinyl polymer, such as at least 2, 5, 10, 20, or 30° C. and/or up to 10, 20, 30, 40, 50, 70, 100, 150, or 200° C. above the decomposition temperature of the vinyl polymer.

The depolymerization method according to the disclosure is particularly effective in that it can achieve relatively high levels of conversion of the original vinyl polymer feed into desired monomers or oligomers corresponding to the original vinyl polymer. Similarly, the depolymerization method can achieve relatively high selectivity values or yields for the desired depolymerization product, whether monomers or oligomers, for example by selecting or controlling one or more of reaction temperature, reaction time, and reaction pressure. Further, the amount of catalyst can be selected to control reaction selectivity. For example, in the case of polystyrene, lower catalyst loadings (e.g., down to about 0.5 or 1 wt.) can favor styrene oligomers or other byproducts over styrene monomer, increasing catalyst loadings (e.g., up to about 5 wt. %) can favor styrene monomer over other products, and further increased catalyst loadings (e.g., above about 5 wt. %) can still effect the desired reaction, but without substantial change in product distribution.

In embodiments, the vinyl polymer initially in the reaction mixture is depolymerized with a conversion of at least 70 wt. %. For example, at least 70, 75, 80, 82, 85, 87, 90, or 92 wt. % and/or up to 85, 88, 90, 92, 95, 98, or 99 wt. % of original vinyl polymer is converted to and/or recovered as the corresponding monomer and/or oligomer products combined, such as in a liquid product phase in the reaction vessel (e.g., in liquid form at ambient temperatures of 5-50° C., 15-35° C., or 20-30° C. after cooling from the high reaction temperature). The foregoing conversion values can reflect the conversion of an individual vinyl polymer (e.g., when multiple different vinyl polymers are initially present in the mixture). Alternatively or additionally, the foregoing conversion values can reflect the collective conversion of all vinyl polymers initially present in the mixture. The remainder of unconverted vinyl polymer can be present after reaction as a solid phase, such as unreacted vinyl polymer (e.g., solidifying or precipitating after cooling to ambient temperatures) and/or carbon-based burning/combustion/etc. residue.

In embodiments, the depolymerization method provides or otherwise results in a liquid product phase containing the corresponding monomer product in an amount of at least 75 wt. % relative to the liquid product phase. As noted above, the liquid product phase can be in liquid form at a temperature in a range of 5-50° C., 15-35° C., or 20-30° C., for example representing an ambient temperature resulting after removal of heat and/or cooling of the product mixture after depolymerization. For example, at least 75, 77, 80, 82, 85, 87, 90, or 92 wt. % and/or up to 85, 88, 90, 92, 95, 98, or 99 wt. % of the liquid product phase can include the corresponding monomer or monomers from the original vinyl polymers. The foregoing ranges can similarly represent monomer yield (relative amount of original vinyl polymer converted to and/or recovered as monomer), for example where there is substantially complete conversion of the vinyl polymer reactant.

In embodiments, the corresponding monomer product (e.g., in the liquid product phase) can be formed with a selectivity of at least 6 relative to other ethylenically unsaturated products, such as ethylenically unsaturated oligomers, ethylenically unsaturated monomers different from the monomer(s) in the original vinyl polymer, etc. For example, the corresponding monomer product can be formed with a selectivity of at least 6, 8, 10, 12, 15, 20, 30, or 40 and/or up to 15, 20, 30, 50, 100, or 200 on a weight/weight basis.

In embodiments, the depolymerization method provides or otherwise results in a liquid product phase containing the corresponding oligomer product in an amount of 25 wt. % or less relative to the liquid product phase. For example, at least 0.1, 1, 2, 3, or 5 wt. % and/or up to 1, 2, 5, 7, 10, 12, 15, 20, or 25 wt. % of the liquid product phase can be one or more corresponding oligomer products with variable lengths, such as dimers, trimers, or higher oligomers, in particular oligomers with 2-4 repeat units. The liquid product phase can further include up to 25 wt. % (with similar subranges as described above) of unsaturated analogs of the corresponding monomers and/or oligomers).

In embodiments, the depolymerization method provides or otherwise results in a liquid depolymerization product (e.g., in liquid form at a temperature in a range of 5-50° C., 15-35° C., or 20-30° C.). The liquid depolymerization product can be an essentially completely depolymerized product such as described above with high monomer yield. Alternatively, the liquid depolymerization product can be a partially depolymerized product that is liquid at room temperature (e.g., containing a sufficient monomer content), but still containing substantial amounts of oligomeric chains. For example, the liquid depolymerization product can include the corresponding monomer product in an amount of 50 wt. % or less relative to the liquid depolymerization product and/or the corresponding oligomer product in an amount of at least 50 wt. % relative to the liquid depolymerization product. More generally, at least 0.1, 1, 2, 3, 5, 10, 15, 20, 25, 30, or 35 wt. % and/or up to 1, 2, 5, 10, 20, 30, 40, or 50 wt. % of the liquid depolymerization product can be the corresponding monomer product. Similarly, at least 50, 60, 70, 75, 80, 85, 90, or 95 wt. % and/or up to 65, 75, 85, 90, 95, 98, 99, or 100 wt. % of the liquid depolymerization product can be the one or more corresponding oligomer products with variable lengths, such as dimers, trimers, or higher oligomers, in particular oligomers with 2-4 repeat units. The liquid depolymerization product can further include up to 25 wt. % (e.g., up to 1, 2, 5, 10, 15, 20, or 25 wt. %) of unsaturated analogs of the corresponding monomers and/or oligomers.

In embodiments, the depolymerization method can further include forming a new or recycled vinyl polymer from the depolymerization product. In particular, when the depolymerization product contains the corresponding monomer product, the monomer can be re-polymerized to form a recycled vinyl polymer. For example, a free radical initiator can be added to the corresponding monomer product (e.g., after separation of the monomer product from other components in the product mixture, such as oligomer products, other monomers, unreacted vinyl polymer or other solids, etc.), and the monomer product can then be heated or reacted under conventional conditions to form the recycled vinyl polymer. The polymerization of the recycled vinyl polymer can be performed in the same or different reaction vessel as for the depolymerization. Free radical initiators are generally known in the art and can include azo initiators (e.g., AIBN or 2,2′-azoisobutyronitrile), organic peroxide initiators (e.g., benzoyl peroxide), aminoxyl, or other initiators.

In some embodiments, the mixture to be depolymerized initially contains substantially only vinyl polymer and catalyst (e.g., as initially fed to a continuous reactor or as initially charged to a batch reactor), for example containing not more than 0.1, 1, 2, 3, or 5 wt. % based on the mixture of components other than the vinyl polymer and catalyst. In some embodiments, the mixture to be depolymerized can initially contain one or more additional components such as additives, impurities, other polymers (e.g., non-vinyl polymers and/or vinyl-backbone polymers without pendant aromatic or ester groups), and/or free-radical initiators, which can be present in a polymer waste stream to be recycled or intentionally added for a processing benefit.

In embodiments, the mixture can include one or more additives and/or impurities in a (combined) amount up to 20 wt. % based on the mixture as a whole, such as at least 0.01, 0.1, 1, 2, or 5 wt. % and/or up to 0.5, 1, 2, 3, 5, 8, 12, 15, or 20 wt. %. Such additional components can include one or more of salts, metal residues, organic impurities or additives, inorganic impurities or additives, moisture, plastic additives, etc. that might be present in a recycled feed as impurities and/or added to the feed mixture as a processing aid.

In embodiments, the mixture can include one or more low-melting thermoplastic polymers in a (combined) amount up to 20 wt. % based on the mixture as a whole, such as at least 0.01, 0.1, 1, 2, or 5 wt. % and/or up to 0.5, 1, 2, 3, 5, 8, 12, 15, or 20 wt. %. Such additional polymers can include polyolefins such as polyethylene (PE, LDPE, HDPE, etc.), polypropylene, etc. that typically have a melting point below about 200° C. or otherwise well below that of the vinyl polymer such that the low-melting thermoplastic polymer melts during heating to provide an initial liquid polymer phase to promote vinyl polymer-catalyst contact and enhance vinyl polymer conversion/yield. The low-melting thermoplastic polymer can be an intentional additive and/or impurity present from a given waste stream to be recycled.

In embodiments, the mixture can include one or more free radical inhibitors or suppressors in a (combined) amount up to 5 wt. % based on the mixture as a whole, such as at least 0.01, 0.1, 1, or 2 wt. % and/or up to 0.5, 1, 2, 3, 4, or 5 wt. %. Free radical suppressors are generally known in the art and can include compounds such as TEMPO (2,2,6,6-tetramethylpiperidine 1-oxyl, 2,2,6,6-tetramethyl-1-piperidinyloxy), N-hydroxysuccinimide, etc.

### EXAMPLES

The following examples illustrate the disclosed methods, but are not intended to limit the scope of any claims thereto.

### Example 1: Depolymerization of Polystyrene

Polystyrene (e.g., in foam form such as STYROFOAM or expanded polystyrene (EPS) foam) is a highly popular packaging plastic material and has a wide range application in electronic goods, electrical appliances, furniture or packaging of food items due to its excellent protective and insulating properties. This example illustrates the ability of the disclosed method to efficiently depolymerize vinyl polymers such as polystyrene using inexpensive metal catalysts such as metal salts, bulk or elemental metal materials, and metal oxides. For example, salt (NaCl)-mediated depolymerization of polystyrene provided styrene monomers at high conversion and yield (above 83%), and the monomers were then polymerized into high molecular weight polystyrene without purification and separation unlike previously reported depolymerization methods in which their low % styrene yield in turn resulted in relatively low molecular weight polystyrene. This example further illustrates the ability of the disclosed method to be used in chemical recycling processes, such as for reprocessing of post-consumer and waste vinyl polymers like polystyrene.

Materials: The following materials were obtained from commercial suppliers and used as received: polystyrene Mw˜35000 (MilliporeSigma), lithium chloride (Fisher Chemical), sodium chloride (Fisher Chemical and Jade Scientific), potassium chloride (ChemPure Brand Chemicals), sodium bromide (Fisher Chemical), sodium iodide (Fisher Chemical), zinc metal (Fisher Chemical), iron metal (˜40 mesh, Spectrum), stainless steel, copper scrubber (Meijer), zinc oxide (Colombia Chemical Industries Inc.), barium oxide (MilliporeSigma), copper oxide (burnt copper wire), graphite powder (MilliporeSigma), TEMPO (2,2,6,6-tetramethylpiperidine 1-oxyl, 2,2,6,6-tetramethyl-1-piperidinyloxy, MilliporeSigma), AIBN (2,2′-azobis(2-methylpropionitrile), MilliporeSigma), N-hydroxysuccinimide (MilliporeSigma), and benzoyl peroxide (MilliporeSigma).

Depolymerization: A 500 mL round bottom flask was charged with 10 grams of polystyrene (PS) pellets (about 2-3 mm diameter). For control samples, either an additional initiator or inhibitor was added to the flask with the PS pellets, or no further components were added to the flask (i.e., pyrolysis depolymerization). For sample according to the disclosure, an additional metal salt, metal, or metal oxide catalyst was added to the flask with the PS pellets. Next the flask was placed on heating mantle and connected to cooling condenser having cold water flow. The condenser was next fit to a schlenk (or receiving) flask). The temperature of the heating mantle was increased to about 420° C. for 45 minutes, after which it was turned off. A yellow-brown product solution of about 8-9 grams was obtained in the receiving flask.

Repolymerization: The receiving flask (100 mL) with product solution therein was separated from the depolymerization set-up, and 2% benzoyl peroxide was added to the product solution, which was placed on a heating plate for 24 hours at 105° C. After this time, 20 mL tetrahydrofuran (THF) were added to solubilize the viscus material formed in the flask. Next, this solution was added to 100 mL of methanol (MeOH) in a dropwise manner. The white precipitate was collected and dried at 100° C. in a vacuum oven about 4-5 hours.

Results: Tables 1˜4 below show the depolymerization results for various control samples and samples according to the disclosure. For each sample, 10 g of PS was used as the initial vinyl polymer amount, and any catalyst or additive to the reaction system is indicated at a wt. % value relative to PS (e.g., 5% NaCl corresponds to 0.5 g NaCl added to 10 g PS). The liquid yield column indicates the total amount of product solution recovered in the receiving flask, and the % styrene content column (analyzed via NMR; not shown) indicates the fraction of the product solution that was styrene monomer.

TABLE 1
Comparative Control Samples

| | | | % Styrene | |
|---|---|---|---|---|
| Sample | PS | Additive | Content | Liquid Yield |
| 1 | 10 g | None (pyrolysis) | 66% | 7.9 g |
| 2 | 10 g | TEMPO (2%) | 71% | 7.8 g |
| 3 | 10 g | AIBN (2%) | 68% | 7.5 g |
| 4 | 10 g | N-Hydroxysuccinimide (2%) | 70% | 7.4 g |
| 5 | 10 g | Benzoyl peroxide (2%) | 63% | 7.6 g |

TABLE 2
Depolymerization Samples with Metal Salt Catalysts

| | | | % Styrene | |
|---|---|---|---|---|
| Sample | PS | Catalyst | Content | Liquid Yield |
| 1 | 10 g | LiCl (Lithium Chloride) 5% | 80% | 6.8 g |
| 2 | 10 g | NaCl (Sodium Chloride) 5% | 82% | 8.0 g |
| 3 | 10 g | KCl (Potassium Chloride) 5% | 81% | 7.9 g |
| 4 | 10 g | NaBr (Sodium Bromide) 5% | 82.5% | 7.4 g |
| 5 | 10 g | NaI (Sodium Iodide) 5% | 79% | 7.6 g |

TABLE 3
Depolymerization Samples with Sodium Chloride Catalyst

| | | | % Styrene | Liquid |
|---|---|---|---|---|
| Sample | PS | Catalyst | Content | Yield |
| 1 | 10 g | NaCl (Sodium Chloride) 1% | 75% | 7.8 g |
| 2 | 10 g | NaCl (Sodium Chloride) 5% | 82% | 8.0 g |
| 3 | 10 g | NaCl (Sodium Chloride) 10% | 82% | 8.0 g |
| 4 | 10 g | NaCl (Sodium Chloride) 25% | 83% | 7.8 g |
| 5 | 10 g | NaCl (Sodium Chloride) 50% | 83% | 7.9 g |
| 6 | 10 g | NaCl (Sodium Chloride) 100% | 84% | 7.9 g |

TABLE 4
Depolymerization Samples with Metal
and Carbon Network Catalysts

| | | | % | |
|---|---|---|---|---|
| | | | Styrene | Liquid |
| Sample | PS | Catalyst/Additive | Content | Yield |
| 1 | 10 g | 5% Zinc Metal (Powder) | 82% | 7.7 g |
| 2 | 10 g | 5% Iron Metal (Powder) | 80% | 7.6 g |
| 3 | 10 g | 5% Stainless Steel (Powder) | 83% | 7.6 g |
| 4 | 10 g | 100% Copper Scrubber (wires) | 81% | 7.7 g |
| 5 | 10 g | 100% Copper Scrubber (wires) + | 81% | 7.7 g |
| | | 5% NaCl | | |
| 6 | 10 g | 100% Copper Oxide (burnt Copper | 84% | 7.9 g |
| | | wire) | | |
| 7 | 10 g | 100% Copper Oxide (burnt Copper | 82% | 8.0 g |
| | | wire) + 5% NaCl | | |
| 8 | 10 g | 5% Zinc Oxide | 80% | 7.8 g |
| 9 | 10 g | 5% Barium oxide | 79% | 7.9 g |
| 10 | 10 g | 5% Graphite Powder | 82.66% | 8.0 g |
| 11 | 10 g | Copper acetate (5 wt %) | 84% | 8.0 g |
| 12 | 10 g | LDPE(5 wt %) + NaCl(5 wt %) | 80% | 8.3 g |
| 13 | 10 g | KRYTOX(5 wt %) + NaCl(5 wt | 81.6 | 8.5 g |
| | | %) | | |
| 14 | 10 g | TEMPO(2 wt %) + NaCl(5 wt %) | 84.66% | 8.3 g |

As shown in Table 1, the addition of an initiator or inhibitor (samples 2-5) did not provide any substantial improvement in depolymerization as compared to pyrolysis (sample 1) without any additive. Table 2 illustrates that a wide variety of alkali metal halide salts provide good catalytic effect for depolymerization with high yields and styrene contents. Table 3 illustrates that high depolymerization yield and styrene content can be obtained for a wide range of catalyst concentrations (using sodium chloride as a representative metal catalyst). Table 4 illustrates that high depolymerization yield and styrene content can be obtained for a wide range of different catalyst materials, including metals, metal oxides, carbon-carbon network catalysts (i.e., graphite as illustrated), and metal salts other than alkali metal halides (i.e., copper acetate as illustrated), as well as in the presence of a low-melting thermoplastic polymer (i.e., LDPE or KRYTOX as illustrated) or radical suppressor (i.e., TEMPO as illustrated).

The product solutions from the pyrolysis control sample (Table 1, sample 1) and the NaCl-catalyzed depolymerization sample (Table 2, sample 2) were diluted 1:1000 in methylene chloride and analyzed via GC/MS for their components. The results are shown in Table 5, and they show that the depolymerization product formed according to the disclosure has a high specificity toward the formation of the desired monomer product corresponding to the original vinyl polymer relative to other monomers and non-polymerizable components (i.e., as reflected by their fractional areas from the GC/MS chromatograms).

TABLE 5
GC/MS Analysis Results of Depolymerization Product

| | Area % | Area % |
|---|---|---|
| Depolymerization Product | (pyrolysis | (NaCl |
| Component | control) | catalyst) |
| Toluene | 5.32% | 7.96% |
| Ethylbenzene | 4.77% | 1.40% |
| Styrene | 67.27% | 83.06% |
| 1-methylethylbenzene | 0.72% | — |
| α-methylstyrene | 9.76% | 4.73% |
| Benzene, 1,1′-(1,3-propanediyl)bis- | 1.20% | — |
| Dimer | 6.61% | 2.85% |
| Trimer | 2.34% | — |

The product solutions from the pyrolysis control sample (Table 1, sample 1) and the NaCl-catalyzed depolymerization sample (Table 2, sample 2) were repolymerized using benzoyl peroxide as described above. Prior to repolymerization, residues such as toluene, ethylbenzene, and unreacted α-methylstyrene were evaporated with the use of a vacuumed line. Further purification was achieved by dissolving the obtained PS in toluene/THF and precipitating the polymer solution into methanol. As a result, 11.5 g of PS was obtained from 14 g of liquid from the NaCl-catalyzed depolymerization sample, and 8.6 g of PS was obtained from 13.5 g of liquid from the pyrolysis control sample. FTIR and NMR analysis (not shown) of the PS from the NaCl-catalyzed depolymerization sample confirmed that the repolymerized PS was essentially similar to commercial available virgin PS. Thermogravimetric analysis (TGA) and differential scanning calorimetry (DSC) analysis similarly confirmed that the NaCl-catalyzed depolymerization sample had properties closer to those of commercial PS as compared to the pyrolysis control sample. The molecular weights of the two PS samples were analyzed via GPC (not shown), and the weight-average molecular weights were about 25,400 g/mol (NaCl-catalyzed depolymerization sample) and 14,300 g/mol (pyrolysis control sample). The lower molecular weight of the control sample was likely due to the presence of remaining α-methylstyrene and other impurities.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

1. A method for depolymerizing a substituted vinyl polymer, the method comprising:
heating a mixture comprising (i) a vinyl polymer comprising one or more vinyl monomer units comprising at least one of a pendant aromatic group and a pendant ester group and (ii) a catalyst comprising at least one of a metal catalyst and a carbon-carbon network catalyst to a temperature above a melting temperature of the vinyl polymer, thereby at least partially depolymerizing the vinyl polymer and forming at least one of a corresponding monomer product and a corresponding oligomer product;
wherein the catalyst is in solid form in the mixture at the temperature to which the mixture is heated.

2. The method of claim 1, wherein the vinyl polymer comprises an aromatic polymer comprising one or more vinyl monomer units comprising a pendant aromatic group.

3. The method of claim 2, wherein the aromatic polymer comprises polystyrene.

4. The method of claim 3, wherein depolymerizing the aromatic polymer comprises forming styrene as the corresponding monomer product.

5. The method of claim 3, wherein depolymerizing the aromatic polymer comprises forming one or more styrene oligomers as the corresponding oligomer product.

6. The method of claim 2, wherein the aromatic polymer comprises a styrene copolymer.

7. The method of claim 2, wherein:
the aromatic polymer comprises polymethylstyrene; and
depolymerizing the aromatic polymer comprises forming at least one of α-methylstyrene as the corresponding monomer product and one or more α-methylstyrene oligomers as the corresponding oligomer product.

8. The method of claim 2, wherein:
the aromatic polymer comprises monomer units resulting from polymerization of monomers according to the general formula CH₂═CR¹R²; and
R¹ and R² independently can be H, substituted or unsubstituted C₁-C₆ linear or branched alkyl groups, substituted or unsubstituted C₂-C₈ linear or branched ester groups, substituted or unsubstituted phenyl groups, substituted or unsubstituted fused polyaromatic groups;
with the proviso that at least one of R¹ and R² includes an aromatic group.

9. The method of claim 1, wherein the vinyl polymer comprises one or more vinyl monomer units comprising a pendant ester group.

10. The method of claim 9, wherein:
the vinyl polymer comprises monomer units resulting from polymerization of monomers according to the general formula CH₂═CR¹R²; and
R¹ and R² independently can be H, substituted or unsubstituted C₁-C₆ linear or branched alkyl groups, substituted or unsubstituted C₂-C₈ linear or branched ester groups, substituted or unsubstituted phenyl groups, substituted or unsubstituted fused polyaromatic groups; and
with the proviso that at least one of R¹ and R² includes an ester group.

11. The method of claim 1, wherein the vinyl polymer is selected from the group consisting of homopolymers, copolymers, and combinations thereof.

12. The method of claim 1, comprising depolymerizing the vinyl polymer with a conversion of at least 70 wt. %.

13. The method of claim 1, wherein depolymerizing the vinyl polymer comprises forming a liquid product phase containing the corresponding monomer product in an amount of at least 75 wt. % relative to the liquid product phase.

14. The method of claim 13, wherein the corresponding monomer product is formed with a selectivity of at least 6 relative to other ethylenically unsaturated products.

15. The method of claim 13, wherein depolymerizing the vinyl polymer comprises forming the corresponding oligomer product in an amount of 25 wt. % or less relative to the liquid product phase.

16. The method of claim 1, wherein depolymerizing the vinyl polymer comprises forming a liquid depolymerization product.

17. The method of claim 16, wherein:
depolymerizing the vinyl polymer comprises forming the corresponding monomer product in an amount of 50 wt. % or less relative to the liquid depolymerization product; and
depolymerizing the vinyl polymer comprises forming the corresponding oligomer product in an amount of at least 50 wt. % relative to the liquid depolymerization product.

18. The method of claim 1, wherein:
depolymerizing the vinyl polymer comprises forming the corresponding monomer product; and
the method further comprises polymerizing the corresponding monomer product to form a recycled vinyl polymer.

19. The method of claim 1, wherein the catalyst is present in the mixture in an amount of 0.5 wt. % to 10 wt. %.

20. The method of claim 1, wherein the catalyst comprises a metal catalyst selected from the group consisting of metal salts, metal oxides, elemental metals, metal alloys, and combinations thereof.

21. The method of claim 20, wherein the metal catalyst comprises an alkali metal halide salt catalyst selected from sodium chloride and potassium chloride.

22. The method of claim 20, wherein the metal catalyst comprises an alkali metal halide salt catalyst comprising: at least one of a lithium cation, a sodium cation, and a potassium cation; and at least one of a fluoride anion, chloride anion, bromide anion, and iodide anion.

23. The method of claim 1, wherein the catalyst comprises a carbon-carbon network catalyst selected from the group consisting of graphite, graphene nanoplatelets, carbon nanotubes, and combinations thereof.

24. The method of claim 1, comprising depolymerizing the vinyl polymer by heating the mixture to a temperature in a range of 250° C. to 400° C.

25. The method of claim 1, comprising depolymerizing the vinyl polymer by heating the mixture to a temperature in a range of 300° C. to 420° C.

26. The method of claim 1, comprising depolymerizing the vinyl polymer by heating the mixture to:
(i) a temperature at least 10° C. above a melting temperature of the vinyl polymer; and
(ii) a temperature at least 50° C. below the decomposition temperature of the vinyl polymer.

27. The method of claim 1, comprising depolymerizing the vinyl polymer by heating the mixture to a temperature up to 200° C. above the decomposition temperature of the vinyl polymer.

28. The method of claim 1, wherein the vinyl polymer is initially in a form selected from foamed materials, un-foamed materials, and combinations thereof.

29. The method of claim 1, wherein the vinyl polymer is present in the mixture in an amount of 80 wt. % to 99 wt. %.

30. The method of claim 1, wherein a combined amount of the vinyl polymer and the catalyst in the mixture is in a range of 80 wt. % to 100 wt. %.

31. The method of claim 1, wherein the mixture further comprises one or more additives or impurities in an amount up to 20 wt. %.

32. The method of claim 1, wherein the mixture further comprises one or more low-melting thermoplastic polymers in an amount up to 20 wt. %.

33. The method of claim 1, wherein the mixture further comprises one or more free radical suppressors in an amount up to 5 wt. %.

34. The method of claim 1, wherein:
depolymerizing the vinyl polymer comprises forming a liquid product phase containing the corresponding monomer product in an amount of at least 75 wt. % relative to the liquid product phase; and
the catalyst comprises a metal catalyst selected from the group consisting of metal salts, metal oxides, elemental metals, metal alloys, and combinations thereof.

35. The method of claim 34, wherein:
the metal catalyst comprises an alkali metal halide salt catalyst selected from sodium chloride and potassium chloride; and
at least one of conditions (I) and (II) is satisfied:
(I) the corresponding monomer product is formed with a selectivity of at least 6 relative to other ethylenically unsaturated products; and
(II) depolymerizing the vinyl polymer comprises forming the corresponding oligomer product in an amount of 25 wt. % or less relative to the liquid product phase.
